(19) United States
(12) Patent Application Publication
(10) Pub. No.: US 2025/0221749 A1
(43) Pub. Date: Jul. 10, 2025

(54) Surgical Fixation System and Related Methods

(71) Applicants: Luis Duarte, San Angelo, TX (US); Shawn Culbertson, Georgetown, TX (US); John Bohenick, Troy, MI (US)

(72) Inventors: Luis Duarte, San Angelo, TX (US); Shawn Culbertson, Georgetown, TX (US); John Bohenick, Troy, MI (US)

(21) Appl. No.: 18/916,636

(22) Filed: Oct. 15, 2024

Related U.S. Application Data
(60) Provisional application No. 63/543,895, filed on Oct. 12, 2023.
(60) Provisional application No. 63/573,629, filed on Apr. 3, 2024.

(51) Int. Cl. A61B 17/84; A61B 17/00

(52) U.S. Cl. CPC A61B 17/844; A61B 17/00234; A61B 2017/00327

(57) ABSTRACT

A surgical fixation system including a bone anchor, rod receiver, compression cap, and stabilization rod is described herein. In some embodiments, the compression cap includes a plurality of ridges configured to interact with a plurality of grooves formed into the stabilization rod. In some embodiments, the fixation system is configured such that upon a first actuation of the compression cap, the compression cap is configured to advance distally within the rod receiver and directly contact the stabilization rod such that at least one rod engaging feature of the plurality of rod engaging features engages at least one groove of the plurality of grooves; and upon a second actuation of the compression cap beyond the first actuation, the compression cap is configured to cause translational movement of the stabilization rod within the rod receiver to effect compression or distraction on the first and second bone segments.

### CROSS-REFERENCES TO RELATED APPLICATIONS

The present application is an nonprovisional application claiming the benefit of priority under 35 U.S.C. § 119(e) from U.S. Provisional Patent Application No. 63/543,895, filed Oct. 12, 2023, and U.S. Provisional Patent Application No. 63/573,629, filed Apr. 3, 2024, the entire contents of which are hereby incorporated by reference into this disclosure as if set forth fully herein.

### FIELD

The present disclosure relates generally to orthopedic surgery, and more particularly to systems and methods that employ anchors to stabilize, maintain spacing between, or couple one or more bone segments.

### BACKGROUND

Spinal pathology is both a common and debilitating condition. Treatment often depends on correcting underlying anatomic problems. The spine is a complex structure and restoring proper function requires both knowledge and technology. Pedicle fixation provides a rigid surgical implant to stabilize adjacent spinal segments in spinal fusion surgery. Pedicle screws have been used to treat spinal disorders, including those caused by spinal cancer, congenital anomalies, trauma, and chronic pain syndromes. Potential complications may include increased pain, infection, or mechanical failure (breakage of the screws or the rods that connect them).

### SUMMARY

The surgical fixation system improves on the current standard of care through new technology and a new revolutionary surgical technique. In some embodiments, it may be desirable to treat one or more bone segments via the surgical fixation system disclosed herein such as in conjunction with other system(s) to encourage bone fusion, stabilize, maintain spacing between, or couple the bone segments, the present invention provides such multi-component bone anchor fixation system.

By way of example only, the surgical fixation system disclosed herein, which may be a referred to herein as a multi-component bone anchor fixation system (or “MBA system”) includes at least one bone anchor, compression cap, stabilization rod, and rod receiver. Generally, a first bone anchor is implanted in a first bone segment to be fused, stabilized, or maintained, a second bone anchor is implanted in a second bone segment to be fused, stabilized, or maintained, a stabilization rod is coupled with the bone anchors by way of rod receivers, and then the stabilization rod is secured in place within the rod receivers by the compression caps. In some embodiments, the fixation system may be configured such that a first actuation of the compression cap advances the compression cap distally within the rod receiver so that the compression cap engages, the stabilization rod. In some embodiments, the fixation system may be configured that a continued tightening of the compression cap by way of a second actuation beyond the first actuation not only tightens and locks the construct, but causes a controlled relative movement between the stabilization rod and the bone anchor to effect compression and/or distraction of the bone segments, depending upon (for example) the direction of rotation of the compression cap and/or directional orientation of grooves or other surface features on the compression cap and/or stabilization rod.

In some embodiments the stabilization rod has a spherical outer surface which enables the stabilization rod to pivot and translate within the rod receiver before being compressed by the compression cap.

In some embodiments, the bone anchor includes head, neck, and elongated shank extending longitudinally from the head and neck in a distal direction.

By way of example only, the head may include a driver interface that may have a mating shape that is complementary to that of a corresponding drive tool in such applications as torque transmission so as to insert the bone anchor into a bone segment. In some embodiments, the head may have a proximal surface that provides an impaction interface for the stabilization rod 16 and may include a tapered ramp extending radially into the driver interface for ease of driver access into the driver interface. In some embodiments, bone anchor may be inserted into a desired position in a bone segment via an impaction tool that acts on the proximal surface of the head. In some embodiments, the head may have a spherical in shape so as to allow articulation once the bone anchor is seated in the rod receiver. This head configuration enables the bone anchor to have a restricted range of motion when used in the assembled fixation system (e.g., with a rod receiver (or “tulip”), rod, and compression cap.

In some embodiments, the elongated shank includes a cylindrical body having a helical thread extending around the body, and a tapered extended tip portion at the distal end of the shank. In some embodiments, the bone anchor may have a linear shape with changing diameter for the body and a tapered diameter for the tapered extended tip portion where the diameter at the distal end of the body is larger than the diameter at the distal end of the tapered tip portion. In some embodiments, the diameter at the distal end of the body may be about 17.5 to 52.5 times larger than diameter at the distal end of the tapered tip portion.

In some embodiments the helical thread of the body and/or tip portion may include a plurality of shelves or ramps curved about a helix. Each shelf or ramp may include a flat ledge portion and an undercut. The flat ledge portion may ease insertion of the bone anchor into a bone segment while the remainder of each ramp, including the undercut and the edge around the undercut may help prevent expulsion of the bone anchor once inserted into a desired position in a bone segment.

In some embodiments, the tip portion may also include a plurality of shelves or ramps with a flat ledge, an undercut, an edge, and a small flat region on the bottom that may also ease insertion of the bone anchor into a bone segment. In some embodiments, the tip section may have a narrower distal end where the helical thread may form an angle of about 10 to 60 degrees and about 20 degrees in an embodiment. In some embodiments, the tip of the bone anchor may be conical with one or more relief sections formed therein. In some embodiments, the relief sections may be cutting flutes formed in the sides of the tip portion. In some embodiments, the tip portion may have three cutting flutes formed therein. The shape of the tip portion may also reduce the force required to inset the bone anchor into a desired position in a bone segment.

In some embodiments, the bone anchor may have a tip having a nonthreaded portion extending between a thread transition point (e.g., the interface between the threaded shank and the nonthreaded portion) and a pointed distal tip. In some embodiments, the nonthreaded portion may have a length dimension with a range of 1 mm to 10 mm. By way of example, the nonthreaded portion is configured to aid entry into bone by way of impaction, for example if a user applies force to the head by way of a mallet or other impaction device, the user may use blunt force to drive the tip of the bone anchor into bone until the threaded portion is within bone, at which point the user may use a driver instrument to rotatably advance the bone anchor further within the bone.

In some embodiments, the tip may include a nonthreaded portion extending between a thread transition point (e.g., the interface between the threaded shank and the nonthreaded portion) and a pointed distal tip, and a fluted portion positioned within the nonthreaded portion. In some embodiments, the nonthreaded portion may have a length dimension with a range of 1 mm to 10 mm. By way of example, the fluted portion provides a drill feature on the tip, which may also be inserted by impaction as described above.

In some embodiments, the head of the bone anchor is configured to mate within a cavity of the rod receiver, such that the bone anchor is rotatable within the cavity until final tightening of the compression cap. In some embodiments, the rod receiver further includes a rod channel positioned between a pair of upstanding arms. In some embodiments, the inner surface of the upstanding arms includes a helical threaded receiving region configured to threadedly mate with the compression cap to enable coupling and advancement of the compression cap within the rod receiver.

In some embodiments, when assembled with the stabilization rod and rod receiver, the compression cap may engage the stabilization rod at about a 90-degree angle at its tip section relative to the stabilization rod.

In some embodiments the compression cap comprises a cylindrical body having a proximal end and a distal end. In some embodiments, the proximal end includes a tool interface which may comprise a recess sized and configured to engage with a driver tool configured to actuate the compression cap. In some embodiments, the distal end comprises a tapered tip and an aperture extending through the locking cap. By way of example, the aperture functions to remove an otherwise pointed tip which increases the surface area of the tapered tip that contacts the stabilization rod upon a first actuation of the compression cap, resulting in a stronger interface between the compression cap and the stabilization rod. In some embodiments, the tapered tip includes a contoured surface configured to engage the stabilization rod and provide a holding force on the stabilization rod after a first actuation of the compression cap. In some embodiments, the contoured surface may comprises a series of ridges, grooves, bumps, troughs, and/or a roughening disposed on the surface. In some embodiments, the cylindrical body includes a helical thread disposed around the body and configured to engage with a complimentary helical threaded receiving region of the rod receiver.

In some embodiments, the contoured surface may comprise a series of ridges arranged in a curved radial pattern. In some embodiments, the ridges may be arranged in a curved radial pattern biased in a clockwise direction. By way of example, this arrangement is configured to engage with the stabilization rod, and in some embodiments, with one or more directional grooves formed in the stabilization rod in a gear or ratchet-like engagement to effect compressive translation of the stabilization rod within the rod receiver upon a second actuation of the compression cap beyond the first actuation, to compress the bone segments upon final tightening. In some embodiments, the ridges may be arranged in a curved radial pattern biased in a counterclockwise direction. By way of example, this arrangement is configured to engage with the stabilization rod, and in some embodiments, with one or more directional grooves formed in the stabilization rod in a gear or ratchet-like engagement to effect distractive translation of the stabilization rod within the rod receiver upon a second actuation of the compression cap beyond the first actuation, to distract the bone segments upon final tightening.

By way of example, in some embodiments the stabilization rods may include a plurality of directional grooves configured to engage with the ridges of the compression cap to enable a gear or ratchet-like engagement and cause translation of the stabilization rod within the rod receiver to effect a compressive force on the bone segments upon final tightening of the compression cap. By way of example, in some embodiments the stabilization rods may include a plurality of directional grooves configured to engage with the ridges of the compression cap to enable a gear or ratchet-like engagement and cause translation of the stabilization rod within the rod receiver to effect a distractive force on the bone segments upon final tightening of the compression cap. By way of example, in some embodiments, the plurality of directional grooves configured to engage with the ridges of the compression cap may be arranged in a linear radial pattern.

By way of example only, when inserted between bone sections, the fixation system may provide substantial retention and anti-expulsion force with the bone segments. In some embodiments, the bone segments may be spinal vertebrae with a disc nucleus located between adjacent vertebrae. In some embodiments, the vertebrae may be lumbar vertebrae and the system including stabilization rod and plurality of bone anchors (e.g., two bone anchors engaging an upper bone segment and two bone anchors engaging the lower, adjacent bone segment) may be configured for insertion from a posterior position (part a lumbar interior fusion procedure).

By way of example, when necessary to remove an inserted bone anchor, it may be ideally moved proximally along a linear axis of the body. A removal tool may be coupled to the driver interface of an inserted bone anchor. The tool may then be used to remove the inserted bone anchor using rotation (e.g., counterclockwise) and linear friction.

As noted, the geometry of the bone anchor may provide greater expulsion strength and reliable cortical vertebral endplate penetration when inserted into vertebra. In some embodiments, the bone anchor outer surface may have scaling to provide increased osteointegration. For example, the bone anchor ramps and undercuts may also grip bone when inserted into thereto. The bone anchor tip structure may enable it to reliably penetrate cortical vertebral endplates without causing nor incurring fracture damage when be inserted into a vertebra. In an embodiment, the bone anchor, stabilization rod, and compression cap may be formed of a biocompatible, substantially radiolucent material or complex of materials.

In some embodiments, the stabilization rod may be formed of a polymer, ceramic, metal, or alloy, including Polyether ether ketone (PEEK) or other member of the polyaryletherketone family, titanium, or cobalt chrome. In some embodiments, the bone anchor may be formed of a metal, alloy, or other osteoconductive material. In some embodiments, the bone anchor may be formed from titanium.

In some embodiments, a method of implanting a surgical fixation system of the present disclosure is described. In some embodiments, a first step of the method is to aim the bone anchor at a target location. In some embodiments, a second step of the method is to insert one or more bone anchors into one or more bone segments using a driver tool. In some embodiments, a third step of the method is to couple the bone anchor to a stabilization rod via a rod receiver. In some embodiments, a fourth step of the method is to insert the compression cap into the rod receiver using a driver. In some embodiments, a fifth step of the method is to apply compression to the fixation system using the driver to apply compressive force to the stabilization rod through the compression cap. In some embodiments, a sixth step of the method is to remove the driver from the field.

As additional description to the embodiments described below, the present disclosure describes the following embodiments.

Embodiment 1 is a surgical fixation system, comprising: a first bone anchor assembly including a first bone anchor, a first rod receiver, and a first compression cap, the first bone anchor including a head configured to couple with the first rod receiver, the first rod receiver including a channel configured to receive a portion of a stabilization rod therein, the first compression cap including an outer threaded surface configured to couple with the first rod receiver and a distal surface including a plurality of rod engaging features positioned thereon; a second bone anchor assembly including a second bone anchor, a second rod receiver, and a second compression cap, the second bone anchor including a head configured to couple with the second rod receiver, the second rod receiver including a channel configured to receive a portion of a stabilization rod therein, the second compression cap including an outer threaded surface configured to couple with the second rod receiver; and a stabilization rod configured for placement within the first and second rod receivers and sized and configured to span a distance between the first and second bone anchor assemblies, the stabilization rod further comprising a plurality of grooves formed therein, the plurality of grooves configured to engage the plurality of rod engaging features of the first compression cap; wherein the first bone anchor assembly is configured for implantation within a first bone segment and the second bone anchor assembly is configured for implantation within a second bone segment different from the first bone segment; wherein upon a first actuation of the first compression cap, the first compression cap is configured to advance distally within the first rod receiver and directly contact the stabilization rod such that at least one rod engaging feature of the plurality of rod engaging features engages at least one groove of the plurality of grooves; and wherein upon a second actuation of the first compression cap beyond the first actuation, the first compression cap is configured to cause translational movement of the stabilization rod within the rod receiver to effect compression or distraction on the first and second bone segments.

Embodiment 2 is the surgical fixation system of embodiment 1, wherein the plurality of grooves on the stabilization rod are oriented such that the grooves are transverse to a longitudinal axis of the stabilization rod.

Embodiment 3 is the surgical fixation system of embodiments 1 or 2, wherein the plurality of grooves on the stabilization rod are oriented such that the grooves are positioned at an oblique angle relative to a longitudinal axis of the stabilization rod.

Embodiment 4 is the surgical fixation system of any of embodiments 1 through 3, wherein plurality of rod engaging features of the first compression cap comprise a plurality of ridges.

Embodiment 5 is the surgical fixation system of any of embodiments 1 through 4, wherein the plurality of ridges is arranged in a curved radial pattern biased in a clockwise direction in which curved ridges extend away from a center of the first compression cap.

Embodiment 6 is the surgical fixation system of any of embodiments 1 through 5, wherein the plurality of ridges arranged in a curved radial pattern biased in a clockwise direction is configured to engage with one or more grooves of the plurality of grooves in a gear or ratchet-like engagement to effect compressive translation of the stabilization rod within the first rod receiver upon a second actuation of the compression cap beyond the first actuation, to compress the first and second bone segments upon final tightening.

Embodiment 7 is the surgical fixation system of any of embodiments 1 through 6, wherein the plurality of ridges is arranged in a curved radial pattern biased in a counterclockwise direction in which curved ridges extend away from a center of the first compression cap.

Embodiment 8 is the surgical fixation system of any of embodiments 1 through 7, wherein the plurality of ridges arranged in a curved radial pattern biased in a counterclockwise direction is configured to engage with one or more grooves of the plurality of grooves in a gear or ratchet-like engagement to effect distractive translation of the stabilization rod within the first rod receiver upon a second actuation of the compression cap beyond the first actuation, to distracdt the first and second bone segments upon final tightening.

Embodiment 9 is the surgical fixation system of any of embodiments 1 through 8, wherein the compression cap comprises a central aperture extending through the compression cap.

Embodiment 10 is the surgical fixation system of any of embodiments 1 through 9, wherein the distal surface of the first compression cap is tapered.

Embodiment 11 is the surgical fixation system of any of embodiments 1 through 10, wherein the second compression cap includes a distal surface including a plurality of rod engaging features positioned thereon, the rod engaging features configured to engage at least one groove of the plurality of grooves of the stabilization rod.

Embodiment 12 is the surgical fixation system of any of embodiments 1 through 11, wherein: upon a first actuation of the second compression cap, the second compression cap is configured to advance distally within the second rod receiver and directly contact the stabilization rod such that at least one rod engaging feature of the plurality of rod engaging features engages at least one groove of the plurality of grooves; and upon a second actuation of the second compression cap beyond the first actuation, the second compression cap is configured to cause translational movement of the stabilization rod within the second rod receiver to effect compression or distraction on the first and second bone segments.

Embodiment 13 is the surgical fixation system of any of embodiments 1 through 12, wherein the plurality of rod engaging features of the second compression cap comprise a plurality of ridges.

Embodiment 14 is the surgical fixation system of any of embodiments 1 through 13, wherein the plurality of ridges is arranged in a curved radial pattern biased in a clockwise direction and is configured to engage with one or more grooves of the plurality of grooves in a gear or ratchet-like engagement to effect compressive translation of the stabilization rod within the second rod receiver upon a second actuation of the compression cap beyond the first actuation, to compress the first and second bone segments upon final tightening.

Embodiment 15 is the surgical fixation system of any of embodiments 1 through 14, wherein the plurality of ridges is arranged in a curved radial pattern biased in a counterclockwise direction and is configured to engage with one or more grooves of the plurality of grooves in a gear or ratchet-like engagement to effect distractive translation of the stabilization rod within the second rod receiver upon a second actuation of the compression cap beyond the first actuation, to distract the first and second bone segments upon final tightening.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIGS. 1-4 are several views of an example of a surgical fixation system of the present disclosure in use to stabilize adjacent bone segments, according to some embodiments;

FIG. 5 is a perspective view of an example of a bone anchor forming part of the surgical fixation system of FIG. 1, according to some embodiments;

FIG. 6 is a perspective view of the bone anchor of FIG. 5, according to some embodiments;

FIG. 7 is a front plan view of the bone anchor of FIG. 5, according to some embodiments;

FIG. 8 is a side plan view of the bone anchor of FIG. 5, according to some embodiments;

FIG. 9 is a perspective view of the bone anchor of FIG. 5 with a nonthreaded tip, according to some embodiments;

FIG. 10 is a plan view of the bone anchor of FIG. 9, according to some embodiments;

FIG. 11 is a perspective view of the bone anchor of FIG. 5 with a nonthreaded tip and a drilling flute, according to some embodiments;

FIG. 12 is a plan view of the bone anchor of FIG. 11, according to some embodiments;

FIG. 13 is a perspective view of an example of a bone anchor of FIG. 5 coupled with a rod receiver forming part of the surgical fixation system of FIG. 1, according to some embodiments;

FIG. 14 is a perspective view of an example of a bone anchor of FIG. 5 coupled with a rod receiver forming part of the surgical fixation system of FIG. 1, according to some embodiments;

FIG. 15 is a front plan view of a bone anchor assembly forming part of the surgical fixation system of FIG. 1, the bone anchor assembly including a bone anchor of FIG. 5 assembled with a rod receiver and an example of a compression cap, according to some embodiments;

FIG. 16 is a side plan view of the bone anchor assembly of FIG. 15, according to some embodiments;

FIG. 17 is a front plan view of the bone anchor assembly of FIG. 15 coupled with a stabilization rod forming part of the surgical fixation system of FIG. 1, according to some embodiments;

FIG. 18 is a top perspective view of an example of a compression cap forming part of the surgical fixation system of FIG. 1, according to some embodiments;

FIG. 19 is a bottom perspective view of the compression cap of FIG. 18, according to some embodiments;

FIG. 20 is a bottom plan view of the compression cap of FIG. 18, according to some embodiments;

FIG. 21 is a side plan view of the compression cap of FIG. 18, according to some embodiments;

FIG. 22 is a bottom plan view of another example of a compression cap forming part of the surgical fixation system of FIG. 1, according to some embodiments;

FIGS. 23-25 are plan views of several examples of stabilization rods with directional grooves formed therein forming part of the surgical fixation system of FIG. 1, according to some embodiments; and

FIG. 26 is a flowchart depicting a method of stabilizing adjacent bone segments using the surgical fixation system of FIG. 1, according to some embodiments.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The surgical fixation system and related methods disclosed herein boasts a variety of inventive features and components that warrant patent protection, both individually and in combination.

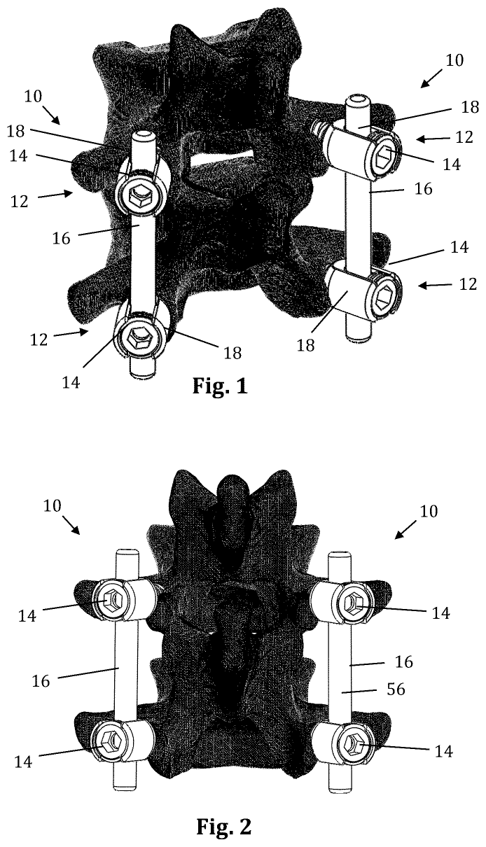

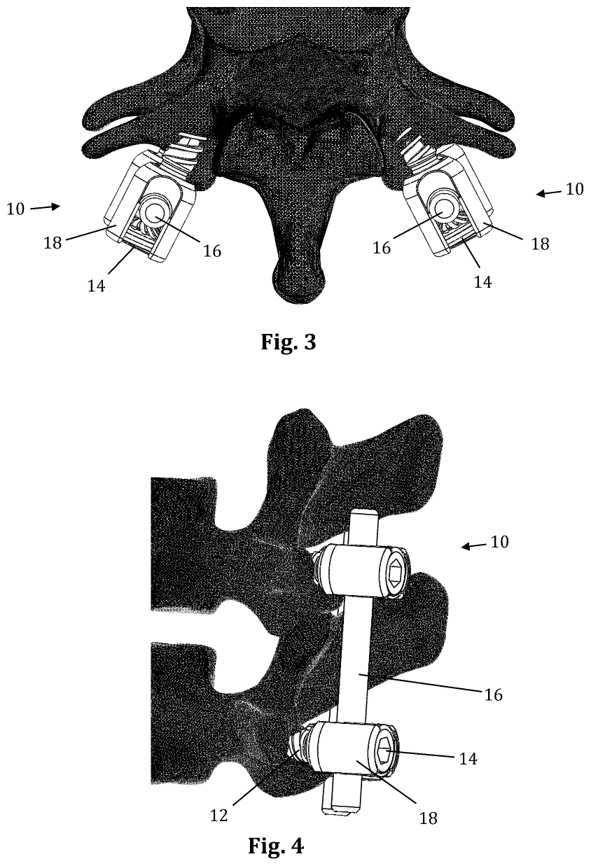

It may be desirable to treat one or more bone segments 5 via one or fixation systems 10 to encourage bone fusion, stabilize, maintain spacing between, or couple the bone segments 5. By way of example only, the surgical fixation system 10, which may be a referred to herein as a multi-component bone anchor fixation system (or “MBA system”) 10 includes at least one bone anchor 12, compression cap 14, stabilization rod 16, and rod receiver 18, as best seen in FIGS. 1-4. Generally, a first bone anchor 12 is implanted in a first bone segment to be fused, stabilized, or maintained, a second bone anchor 12 is implanted in a second bone segment to be fused, stabilized, or maintained, a stabilization rod 16 is coupled with the bone anchors 12 by way of rod receivers 18, and then the stabilization rod 16 is secured in place within the rod receivers 18 by the compression caps 14. In some embodiments, the fixation system 10 may be configured such that a first actuation of the compression cap 14 advances the compression cap 14 distally within the rod receiver 18 so that the compression cap 14 engages, the stabilization rod 16. In some embodiments, the fixation system 10 may be configured that a continued tightening of the compression cap 14 by way of a second actuation beyond the first actuation not only tightens the construct, but enables a controlled relative movement between the stabilization rod 16 and the bone anchor 12 to effect compression and/or distraction of the bone segments 5, depending upon (for example) the direction of rotation of the compression cap and/or directional orientation of grooves or other surface features on the compression cap 14 and/or stabilization rod 16.

In some embodiments the stabilization rod 16 has a spherical outer surface 56 which enables the stabilization rod 16 to pivot and translate within the rod receiver 18 before being compressed by the compression cap 14.

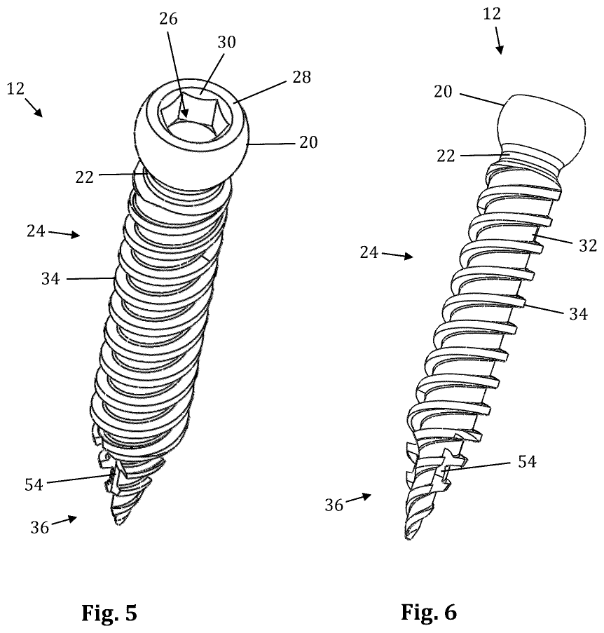

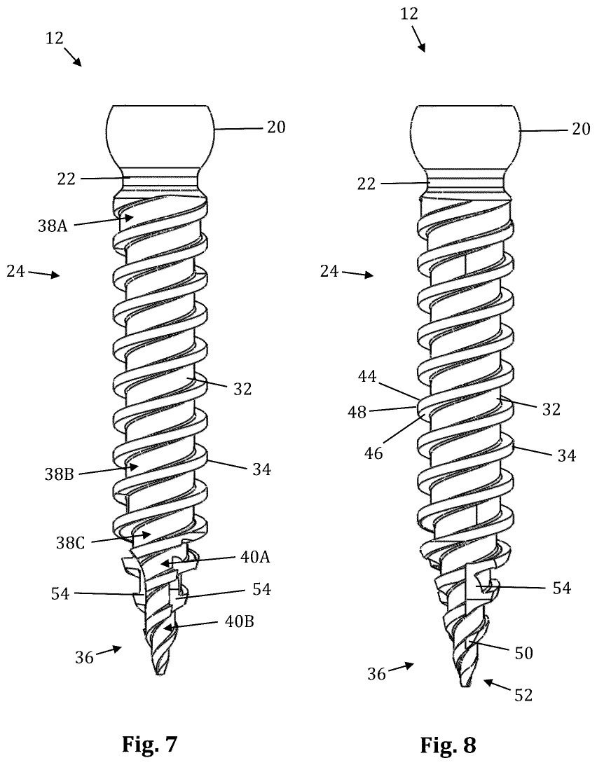

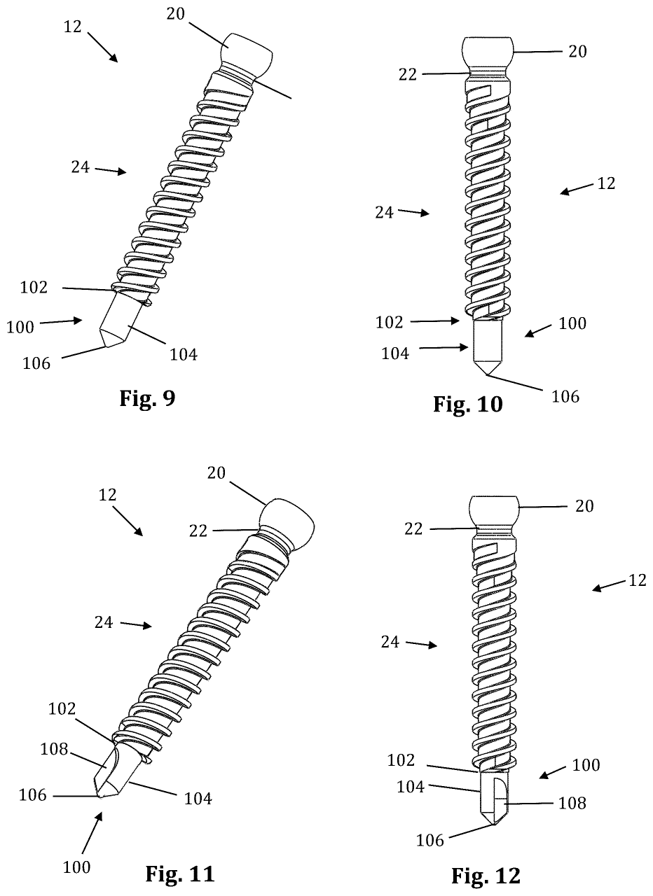

By way of example, FIG. 5 is a simplified isometric front drawing of an example bone anchor 12 forming part of the fixation system 10 according to some embodiments. FIG. 6 is a simplified rear drawing of the bone anchor 12 according to some embodiments. FIG. 7 is a simplified front view of the bone anchor 12 according to some embodiments. FIG. 8 is a simplified side view of the bone anchor 12 according to some embodiments. FIG. 9 is an isometric view of the bone anchor 12 with a nonthreaded tip 100 according to some embodiments. FIG. 10 is a plan view of the bone anchor 12 with a nonthreaded tip 100 according to some embodiments. FIG. 11 is an isometric view of the bone anchor 12 with a nonthreaded tip 100 and a fluted portion 108 according to some embodiments. FIG. 12 is plan view of the bone anchor 12 with a nonthreaded tip 100 and a fluted portion 108 according to some embodiments.

In some embodiments, the bone anchor 12 includes head 20, neck 22, and elongated shank 24 extending longitudinally from the head 20 and neck 22 in a distal direction.

By way of example only, the head 20 may include a driver interface 26 that may have a mating shape that is complementary to that of a corresponding drive tool in such applications as torque transmission so as to insert the bone anchor 12 into a bone segment 5. In some embodiments, the head 20 may have a proximal surface 28 that provides an impaction interface for the stabilization rod 16 and may include a tapered ramp 30 extending radially into the driver interface 26 for ease of driver access into the driver interface 26. In some embodiments, a multi-component bone anchor (MBA) 10 may be inserted into a desired position in a bone segment 5 via an impaction tool that acts on the proximal surface 28 of the head 20. In some embodiments, the head 20 may have a spherical in shape so as to allow articulation once the bone anchor 12 is seated in the rod receiver 18. This head 20 configuration enables the bone anchor to have a restricted range of motion when used in the assembled MBA system 10 (e.g., with a rod receiver 18 (or “tulip”), rod 16, and compression cap 14).

In some embodiments, the elongated shank 24 includes a cylindrical body 32 having a helical thread 34 extending around the body 32, and a tapered extended tip portion 36 at the distal end of the shank 24. As shown in FIGS. 5-8, the bone anchor 12 may have a linear shape with changing diameter 38A-38C for the body 32 and a tapered diameter 40A-40B for the tapered extended tip portion 36 where the diameter 38C at the distal end of the body 32 is larger than the diameter 40B at the distal end of the tapered tip portion 36. In some embodiments, the diameter 38C may be about 17.5 to 52.5 times larger than diameter 40B.

In some embodiments the helical thread 34 of the body 32 and/or tip portion 36 may include a plurality of shelves or ramps 42 curved about a helix. Each shelf or ramp 42 may include a flat ledge portion 44 and an undercut 46. The flat ledge portion 44 may ease insertion of the bone anchor 12 into a bone segment 5 while the remainder of each ramp 42, including the undercut 46 and the edge 48 around the undercut 46 may help prevent expulsion of the bone anchor 12 once inserted into a desired position in a bone segment 5.

In some embodiments, the tip portion 36 may also include a plurality of shelves or ramps 42 with a flat ledge 44, an undercut 46, an edge 48, and a small flat region 50 on the bottom that may also ease insertion of the bone anchor 12 into a bone segment 5. In some embodiments, the tip section 36 may have a narrower distal end 52 where the helical thread 34 may form an angle of about 10 to 60 degrees and about 20 degrees in an embodiment. In some embodiments, the tip 36 of the bone anchor 12 may be conical with one or more relief sections 54 formed therein. In some embodiments, the relief sections 54 may be cutting flutes formed in the sides of the tip portion 36. In some embodiments, the tip portion 36 may have three cutting flutes 54 formed therein. The shape of the tip portion 36 may also reduce the force required to inset the bone anchor 12 into a desired position in a bone segment 5.

In some embodiments, as illustrated in FIGS. 9-10, the bone anchor 12 may have a tip 100 having a nonthreaded portion 104 extending between a thread transition point 102 (e.g., the interface between the threaded shank and the nonthreaded portion 104) and a pointed distal tip 106. In some embodiments, the nonthreaded portion may have a length dimension with a range of 1 mm to 10 mm. By way of example, the nonthreaded portion 104 is configured to aid entry into bone by way of impaction, for example if a user applies force to the head 20 by way of a mallet or other impaction device, the user may use blunt force to drive the tip 100 of the bone anchor 12 into bone until the threaded portion 34 is within bone, at which point the user may use a driver instrument to rotatably advance the bone anchor 12 further within the bone.

In some embodiments, as illustrated by way of example in FIGS. 11-12, the tip 100 may include a nonthreaded portion 104 extending between a thread transition point 102 (e.g., the interface between the threaded shank and the nonthreaded portion 104) and a pointed distal tip 106, and a fluted portion 108 positioned within the nonthreaded portion 104. In some embodiments, the nonthreaded portion may have a length dimension with a range of 1 mm to 10 mm. By way of example, the fluted portion 108 provides a drill feature on the tip 100, which may also be inserted by impaction as described above.

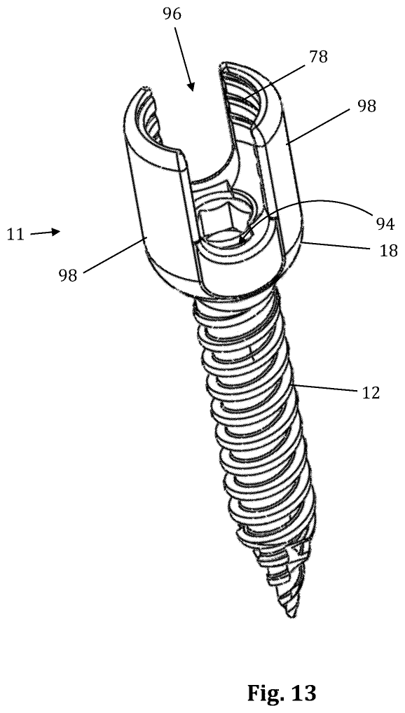

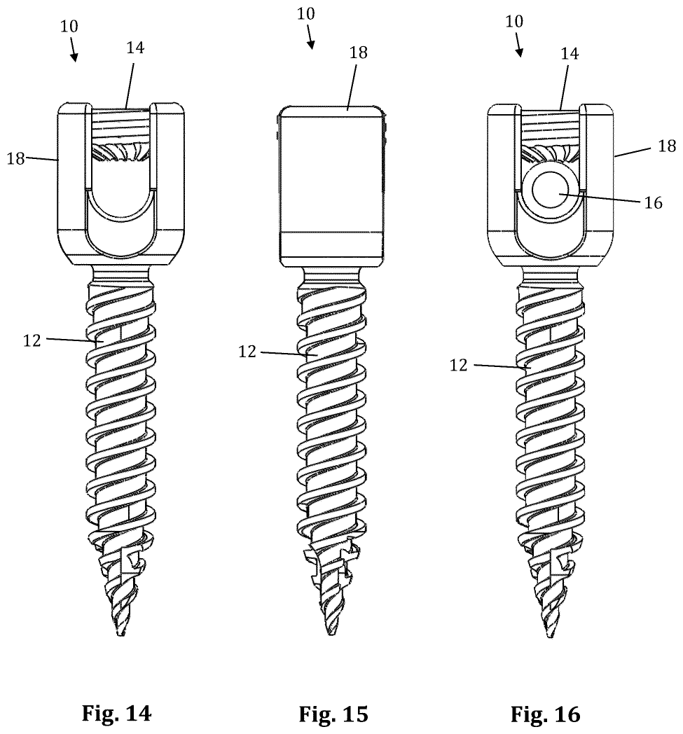

FIGS. 13-16 illustrate an example of a bone anchor assembly 11 configured for use with the surgical fixation system 10 of the present disclosure. In some embodiments, the head 22 of the bone anchor 12 is configured to mate within a cavity 94 of the rod receiver 18, such that the bone anchor 12 is rotatable within the cavity 94 until final tightening of the compression cap 14. In some embodiments, the rod receiver 18 further includes a rod channel 96 positioned between a pair of upstanding arms 98. In some embodiments, the inner surface of the upstanding arms 98 includes a helical threaded receiving region 78 configured to threadedly mate with the compression cap 14 to enable coupling and advancement of the compression cap 14 within the rod receiver 18.

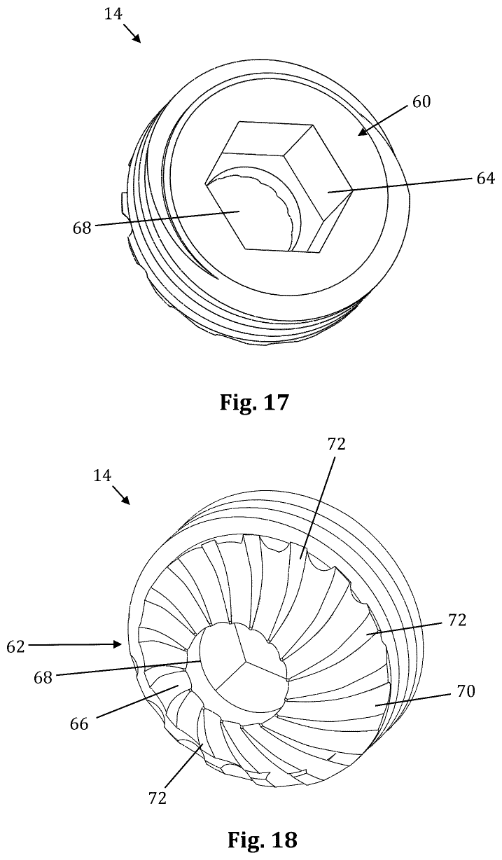

In some embodiments, for example as shown in FIG. 16, when assembled with the stabilization rod 16 and rod receiver 18, the compression cap 14 may engage the stabilization rod 16 at about a 90-degree angle at its tip section 66 relative to the stabilization rod 16.

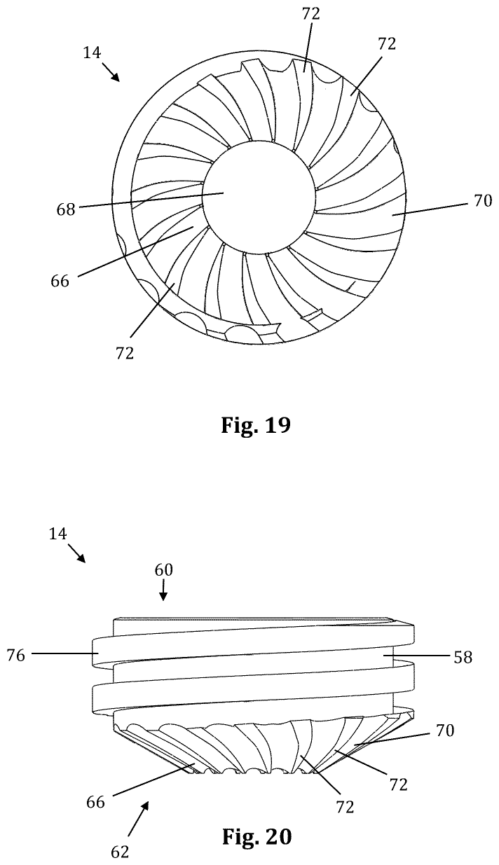

As shown in FIGS. 17-20, in some embodiments the compression cap 14 comprises a cylindrical body 58 having a proximal end 60 and a distal end 62. In some embodiments, the proximal end 60 includes a tool interface 64 which may comprise a recess sized and configured to engage with a driver tool configured to actuate the compression cap 14. In some embodiments, the distal end 62 comprises a tapered tip 66 and an aperture 68 extending through the locking cap 14. By way of example, the aperture 68 functions to remove an otherwise pointed tip which increases the surface area of the tapered tip 66 that contacts the stabilization rod 16 upon a first actuation of the compression cap 18, resulting in a stronger interface between the compression cap 18 and the stabilization rod 16. In some embodiments, the tapered tip 66 includes a contoured surface 70 configured to engage the stabilization rod 16 and provide a holding force on the stabilization rod 16 after a first actuation of the compression cap 14. In some embodiments, the contoured surface 70 may comprises a series of ridges, grooves, bumps, troughs, and/or a roughening disposed on the surface 70. In some embodiments, the cylindrical body 58 includes a helical thread 76 disposed around the body 58 and configured to engage with a complimentary helical threaded receiving region 78 of the rod receiver 18 (FIG. 13).

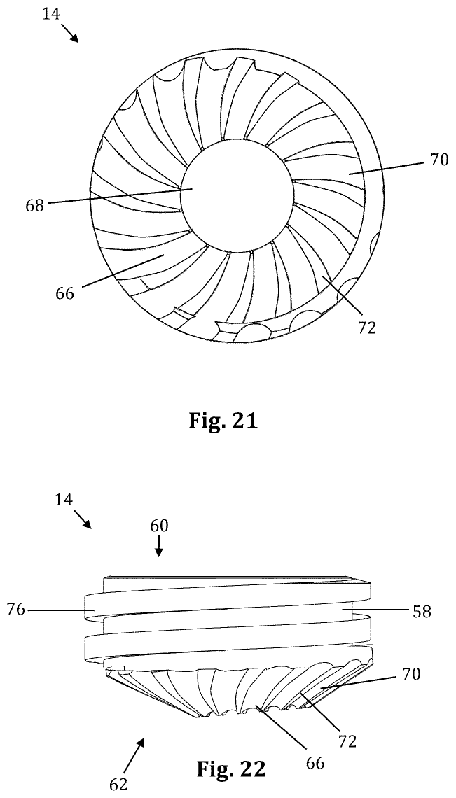

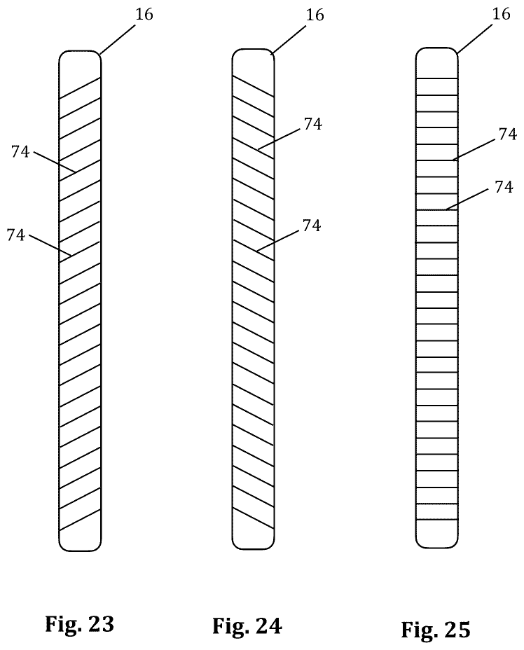

In some embodiments, the contoured surface 70 may comprise a series of ridges 72 arranged in a curved radial pattern. In some embodiments, for example as shown in FIG. 19, the ridges 72 may be arranged in a curved radial pattern biased in a clockwise direction. By way of example, this arrangement is configured to engage with the stabilization rod 16, and in some embodiments, with one or more directional grooves 74 formed in the stabilization rod 16 (See, e.g., FIGS. 23-25) in a gear or ratchet-like engagement to effect compressive translation of the stabilization rod 16 within the rod receiver 18 upon a second actuation of the compression cap 14 beyond the first actuation, to compress the bone segments 5 upon final tightening. In some embodiments, for example as shown in FIGS. 21, the ridges 72b may be arranged in a curved radial pattern biased in a counterclockwise direction. By way of example, this arrangement is configured to engage with the stabilization rod 16, and in some embodiments, with one or more directional grooves 74 formed in the stabilization rod 16 (See, e.g., FIGS. 23-25) in a gear or ratchet-like engagement to effect distractive translation of the stabilization rod 16 within the rod receiver 18 upon a second actuation of the compression cap 14 beyond the first actuation, to distract the bone segments 5 upon final tightening.

FIGS. 23-25 illustrate examples of stabilization rods 16 having directional grooves 74 formed therein, according to some embodiments. By way of example, FIG. 23 illustrates a plurality of directional grooves 74 configured to engage with the ridges 72 of the compression cap 14 shown by way of example in FIG. 19 to enable a gear or ratchet-like engagement and cause translation of the stabilization rod 16 within the rod receiver 18 to effect a compressive force on the bone segments 5 upon final tightening of the compression cap 14. By way of example, FIG. 24 illustrates a plurality of directional grooves 74 configured to engage with the ridges 72 of the compression cap 14 shown by way of example in FIG. 21 to enable a gear or ratchet-like engagement and cause translation of the stabilization rod 16 within the rod receiver 18 to effect a distractive force on the bone segments 5 upon final tightening of the compression cap 14. By way of example, FIG. 25 illustrates a plurality of directional grooves 74 configured to engage with the ridges 72 of the compression cap 14 that may be arranged in a linear radial pattern.

By way of example only, when inserted between bone sections 5 as shown in FIGS. 1-4, the fixation system 10 may provide substantial retention and anti-expulsion force with the bone segments 5. In some embodiments, the bone segments 5 may be spinal vertebrae with a disc nucleus located between adjacent vertebrae 5. In some embodiments, the vertebrae may be lumbar vertebrae and the system 10 including stabilization rod 16 and plurality of bone anchors 12 (e.g., two bone anchors 12 engaging an upper bone segment 5 and two bone anchors 12 engaging the lower, adjacent bone segment 5) may be configured for insertion from a posterior position (part a lumbar interior fusion procedure).

By way of example, when necessary to remove an inserted bone anchor 12, it may be ideally moved proximally along a linear axis of the body 32. A removal tool may be coupled to the driver interface 26 of an inserted bone anchor 12. The tool may then be used to remove the inserted bone anchor 12 using rotation (e.g., counterclockwise) and linear friction.

As noted, the geometry of the bone anchor 12 may provide greater expulsion strength and reliable cortical vertebral endplate penetration when inserted into vertebra 5. In some embodiments, the bone anchor 12 outer surface may have scaling to provide increased osteointegration. For example, the bone anchor 12 ramps 42 and undercuts 44 may also grip bone when inserted into thereto. The bone anchor 12 tip 36 structure may enable it to reliably penetrate cortical vertebral endplates without causing nor incurring fracture damage when be inserted into a vertebra 5. In an embodiment, the bone anchor 12, stabilization rod 16, and compression cap 14 may be formed of a biocompatible, substantially radiolucent material or complex of materials.

In some embodiments, the stabilization rod 16 may be formed of a polymer, ceramic, metal, or alloy, including Polyether ether ketone (PEEK) or other member of the polyaryletherketone family, titanium, or cobalt chrome. In some embodiments, the bone anchor 12 may be formed of a metal, alloy, or other osteoconductive material. In some embodiments, the bone anchor 12 may be formed from titanium.

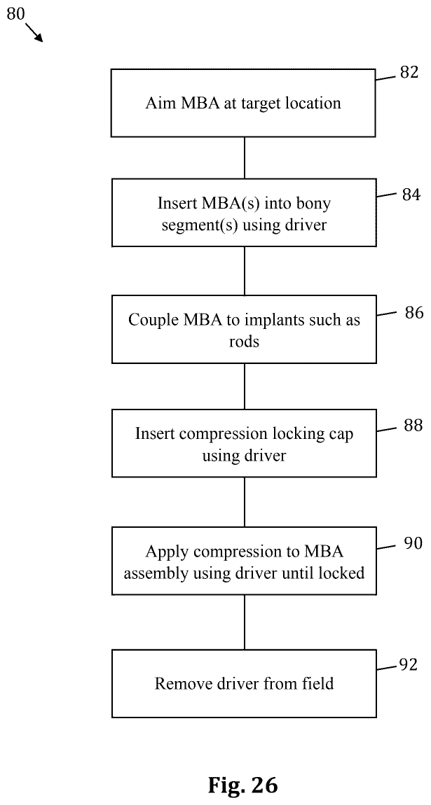

FIG. 26 illustrates a method 80 of implanting a fixation system 10 of the present disclosure, according to some embodiments. In some embodiments, a first step 82 is to aim the bone anchor 12 at a target location. In some embodiments, a second step 84 is to insert one or more bone anchors 12 into one or more bone segments 5 using a driver tool. In some embodiments, a third step 86 is to couple the bone anchor 12 to a stabilization rod 16 via a rod receiver 18. In some embodiments, a fourth step 88 is to insert the compression cap 14 into the rod receiver 18 using a driver. In some embodiments, a fifth step 90 is to apply compression to the fixation system 10 using the driver to apply compressive force to the stabilization rod 16 through the compression cap 14. In some embodiments, a sixth step 92 is to remove the driver from the field.

The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A surgical fixation system, comprising:
a first bone anchor assembly including a first bone anchor, a first rod receiver, and a first compression cap, the first bone anchor including a head configured to couple with the first rod receiver, the first rod receiver including a channel configured to receive a portion of a stabilization rod therein, the first compression cap including an outer threaded surface configured to couple with the first rod receiver and a distal surface including a plurality of rod engaging features positioned thereon;
a second bone anchor assembly including a second bone anchor, a second rod receiver, and a second compression cap, the second bone anchor including a head configured to couple with the second rod receiver, the second rod receiver including a channel configured to receive a portion of a stabilization rod therein, the second compression cap including an outer threaded surface configured to couple with the second rod receiver; and
a stabilization rod configured for placement within the first and second rod receivers and sized and configured to span a distance between the first and second bone anchor assemblies, the stabilization rod further comprising a plurality of grooves formed therein, the plurality of grooves configured to engage the plurality of rod engaging features of the first compression cap;
wherein the first bone anchor assembly is configured for implantation within a first bone segment and the second bone anchor assembly is configured for implantation within a second bone segment different from the first bone segment;
wherein upon a first actuation of the first compression cap, the first compression cap is configured to advance distally within the first rod receiver and directly contact the stabilization rod such that at least one rod engaging feature of the plurality of rod engaging features engages at least one groove of the plurality of grooves; and
wherein upon a second actuation of the first compression cap beyond the first actuation, the first compression cap is configured to cause translational movement of the stabilization rod within the rod receiver to effect compression or distraction on the first and second bone segments.

2. The surgical fixation system of claim 1, wherein the plurality of grooves on the stabilization rod are oriented such that the grooves are transverse to a longitudinal axis of the stabilization rod.

3. The surgical fixation system of claim 1, wherein the plurality of grooves on the stabilization rod are oriented such that the grooves are positioned at an oblique angle relative to a longitudinal axis of the stabilization rod.

4. The surgical fixation system of claim 1, wherein plurality of rod engaging features of the first compression cap comprise a plurality of ridges.

5. The surgical fixation system of claim 4, wherein the plurality of ridges is arranged in a curved radial pattern biased in a clockwise direction in which curved ridges extend away from a center of the first compression cap.

6. The surgical fixation system of claim 4, wherein the plurality of ridges arranged in a curved radial pattern biased in a clockwise direction is configured to engage with one or more grooves of the plurality of grooves in a gear or ratchet-like engagement to effect compressive translation of the stabilization rod within the first rod receiver upon a second actuation of the compression cap beyond the first actuation, to compress the first and second bone segments upon final tightening.

7. The surgical fixation system of claim 4, wherein the plurality of ridges is arranged in a curved radial pattern biased in a counterclockwise direction in which curved ridges extend away from a center of the first compression cap.

8. The surgical fixation system of claim 7, wherein the plurality of ridges arranged in a curved radial pattern biased in a counterclockwise direction is configured to engage with one or more grooves of the plurality of grooves in a gear or ratchet-like engagement to effect distractive translation of the stabilization rod within the first rod receiver upon a second actuation of the compression cap beyond the first actuation, to distracdt the first and second bone segments upon final tightening.

9. The surgical fixation system of claim 1, wherein the compression cap comprises a central aperture extending through the compression cap.

10. The surgical fixation system of claim 1, wherein the distal surface of the first compression cap is tapered.

11. The surgical fixation system of claim 1, wherein the second compression cap includes a distal surface including a plurality of rod engaging features positioned thereon, the rod engaging features configured to engage at least one groove of the plurality of grooves of the stabilization rod.

12. The surgical fixation system of claim 11, wherein:
upon a first actuation of the second compression cap, the second compression cap is configured to advance distally within the second rod receiver and directly contact the stabilization rod such that at least one rod engaging feature of the plurality of rod engaging features engages at least one groove of the plurality of grooves; and
upon a second actuation of the second compression cap beyond the first actuation, the second compression cap is configured to cause translational movement of the stabilization rod within the second rod receiver to effect compression or distraction on the first and second bone segments.

13. The surgical fixation system of claim 12, wherein the plurality of rod engaging features of the second compression cap comprise a plurality of ridges.

14. The surgical fixation system of claim 13, wherein the plurality of ridges is arranged in a curved radial pattern biased in a clockwise direction and is configured to engage with one or more grooves of the plurality of grooves in a gear or ratchet-like engagement to effect compressive translation of the stabilization rod within the second rod receiver upon a second actuation of the compression cap beyond the first actuation, to compress the first and second bone segments upon final tightening.

15. The surgical fixation system of claim 13, wherein the plurality of ridges is arranged in a curved radial pattern biased in a counterclockwise direction and is configured to engage with one or more grooves of the plurality of grooves in a gear or ratchet-like engagement to effect distractive translation of the stabilization rod within the second rod receiver upon a second actuation of the compression cap beyond the first actuation, to distract the first and second bone segments upon final tightening.
